(19) United States
(12) Patent Application Publication
(10) Pub. No.: US 2026/0334821 A1
(43) Pub. Date: Oct. 29, 2026

(54) MEASUREMENT DEVICE AND MEASUREMENT METHOD

(71) Applicants: SONY SEMICONDUCTOR SOLUTIONS CORPORATION, Kanagawa (JP); SONY GROUP CORPORATION, Tokyo (JP)

(72) Inventors: Fumitaka KONDO, Kanagawa (JP); Ken YAMAMOTO, Kanagawa (JP); Kei NAKAGAWA, Kanagawa (JP); Ayaka HIYAMA, Tokyo (JP)

(73) Assignees: SONY SEMICONDUCTOR SOLUTIONS CORPORATION, Kanagawa (JP); SONY GROUP CORPORATION, Tokyo (JP)

(21) Appl. No.: 19/110,963

(22) PCT Filed: Sep. 12, 2023
(86) PCT No.: PCT/JP2023/033140

§ 371 Date: Mar. 12, 2025

(30) Foreign Application Priority Data
Sep. 30, 2022 (JP) 2022-158497

(51) Int. Cl. A61B 5/145; A61B 5/00; A61B 5/0537

(52) U.S. Cl. CPC A61B 5/14532; A61B 5/0537; A61B 5/681

(57) ABSTRACT

The present technology relates to a measurement device that enables accurate measurement of the concentration of glucose (glucose sugar) contained in a measurement target, and a measurement method. The glucose concentration in the measurement target is calculated on the basis of the complex dielectric constant of the measurement target and the moisture content of the measurement target. The moisture content can be calculated on the basis of an incident signal as an electromagnetic wave that has entered the measurement target, and a reflected signal reflected from the measurement target with respect to the incident signal.

### TECHNICAL FIELD

The present technology relates to a measurement device and a measurement method, and more particularly, to a measurement device that enables accurate measurement of the concentration of glucose (glucose sugar) contained in a measurement target, and a measurement method.

### BACKGROUND ART

Regarding non-invasive blood glucose level sensor, sensors using an infrared laser, microwaves, and millimeter waves have been brought to production and been studied. By a technology that is used in a today's non-invasive blood glucose level sensor, there is a problem in that the accuracy drops depending on the proportion of bulk water in the body or outside the body (such as sweat). Patent Document 1 discloses a technique for measuring a component concentration with high accuracy by reducing the influence of the moisture content of the measurement target when the component concentration is measured with dielectric spectroscopy.

### CITATION LIST

### Patent Document

Patent Document 1: Japanese Patent Application Laid-Open No. 2019-190896

### SUMMARY OF THE INVENTION

### Problems to be Solved by the Invention

In Patent Document 1, the influence of the moisture content of the measurement target on the complex dielectric constant spectrum is reduced by a process called normalization of the complex dielectric constant spectrum, and thus, the influence of the moisture content of the measurement target on a component concentration as a measurement result is reduced. However, it is difficult to sufficiently reduce the influence of the moisture content of the measurement target on the complex dielectric constant spectrum simply by normalization of the complex dielectric constant spectrum.

The present technology has been made in view of such circumstances, and enables accurate measurement of the concentration of glucose (glucose sugar) contained in a measurement target.

### Solutions to Problems

A measurement device according to a first aspect of the present technology is a measurement device including a processing unit that calculates the glucose concentration in a measurement target, on the basis of the complex dielectric constant of the measurement target and the moisture content of the measurement target.

A measurement method according to the first aspect of the present technology is a measurement method implemented by a processing unit of a measurement device including the processing unit, the measurement method including calculating the glucose concentration in a measurement target, on the basis of the complex dielectric constant of the measurement target and the moisture content of the measurement target.

In the measurement device and by the measurement method according to the first aspect of the present technology, the glucose concentration in a measurement target is calculated on the basis of the complex dielectric constant of the measurement target and the moisture content of the measurement target.

A measurement device according to a second aspect of the present technology is a measurement device including: an RF circuit that generates an incident signal as an electromagnetic wave to enter a measurement target, and receives the incident signal and a reflected signal reflected from the measurement target with respect to the incident signal; an AD converter that converts the incident signal and the reflected signal received by the RF circuit into digital data; a transform unit that performs Fourier transform on the incident signal and the reflected signal converted into the digital data by the AD converter; and an S-parameter calculation unit that calculates scattering parameters (S parameters), on the basis of the incident signal and the reflected signal subjected to the Fourier transform by the transform unit.

A measurement method according to the second aspect of the present technology is a measurement method implemented by a measurement device including: an RF circuit; an AD converter; a transform unit; and an S-parameter calculation unit, the measurement method including: the RF circuit generating an incident signal as an electromagnetic wave to enter a measurement target, and receiving the incident signal and a reflected signal reflected from the measurement target with respect to the incident signal; the AD converter converting the incident signal and the reflected signal received by the RF circuit into digital data; the transform unit performing Fourier transform on the incident signal and the reflected signal converted into the digital data by the AD converter; and the S-parameter calculation unit calculating S parameters on the basis of the incident signal and the reflected signal subjected to the Fourier transform by the transform unit.

In the measurement device and by the measurement method according to the second aspect of the present technology: an incident signal as an electromagnetic wave to enter a measurement target is generated, and the incident signal and a reflected signal reflected from the measurement target with respect to the incident signal are received; the incident signal and the reflected signal are converted into digital data; Fourier transform is performed on the incident signal and the reflected signal converted into the digital data; and S parameters are calculated on the basis of the incident signal and the reflected signal subjected to the Fourier transform.

### BRIEF DESCRIPTION OF DRAWINGS

FIG. 1 is a block diagram illustrating an example schematic configuration of a first embodiment of a measurement device to which the present technology is applied.

FIG. 2 is a configuration diagram illustrating an example configuration of Example 1 of a measurement device.

FIG. 3 is a configuration diagram illustrating an example configuration of Example 2 of a measurement device.

FIG. 4 is a configuration diagram illustrating an example configuration of Example 3 of a measurement device.

FIG. 5 is a block diagram illustrating an example schematic configuration of a second embodiment of a measurement device to which the present technology is applied.

FIG. 6 is a flowchart illustrating an example of processing procedures to be carried out by a measurement device to which the present technology is applied.

FIG. 7 is a configuration diagram illustrating an example of details of a dielectric spectroscopy unit in Example 2 in FIG. 3.

FIG. 8 is a diagram illustrating an example arrangement 1 of probes.

FIG. 9 is a diagram illustrating an example arrangement 2 of probes.

FIG. 10 is a diagram illustrating an example in which the example arrangement 1 of probes is adopted.

FIG. 11 is a diagram illustrating an example in which the example arrangement 2 of probes is adopted.

FIG. 12 is a diagram illustrating a specific example of configurations of a dielectric spectroscopy unit and a signal processing unit of a measurement device.

### MODE FOR CARRYING OUT THE INVENTION

The following is a description of embodiments of the present technology, with reference to the drawings.

### First Embodiment of a Measurement Device to Which the Present Technology is Applied

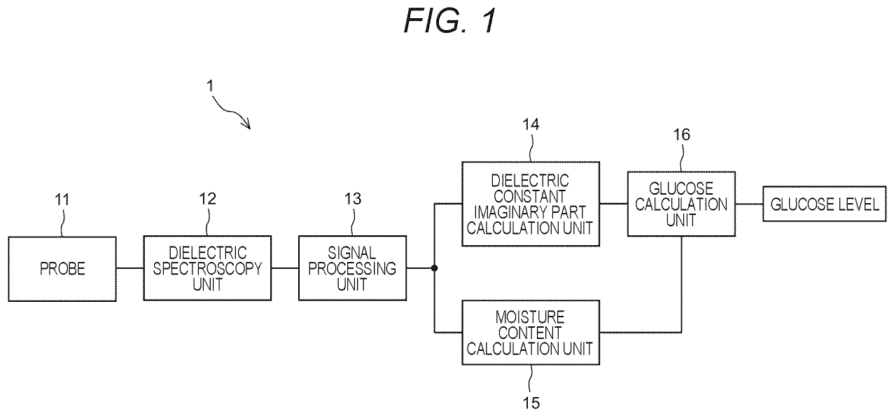

FIG. 1 is a block diagram illustrating an example schematic configuration of a first embodiment of a measurement device to which the present technology is applied.

In FIG. 1, a measurement device 1 is a measurement device according to the first embodiment, and includes a configuration of a vector network analyzer (VNA), for example. As is well known, a vector network analyzer is a device that measures scattering parameters (S parameters) of a measurement target.

The measurement device 1 includes a probe 11, a dielectric spectroscopy unit 12, a signal processing unit 13, a dielectric constant imaginary part calculation unit 14, a moisture content calculation unit 15, and a glucose calculation unit 16. The probe 11 comprehensively represents probes forming each port of one or a plurality of ports of the vector network analyzer. The probe 11 irradiates (enters) the measurement target with electromagnetic waves in a micro-to millimeter-wave band (1 to 9 GHz, for example) supplied from the dielectric spectroscopy unit 12 (these electromagnetic waves will be hereinafter referred to simply as the electromagnetic waves), and supplies (transmits) the electromagnetic waves reflected or transmitted (transferred) from the measurement target to the dielectric spectroscopy unit 12. The probe 11 may be formed with a coaxial probe, a waveguide, a microstrip line, a coplanar line, or the like as an electromagnetic wave transmission line. Note that at least one probe of the comprehensively represented probe 11 forms a port (a port 1) through which the electromagnetic waves enter the measurement target. The probe of the port 1 transmits an electromagnetic wave (also referred to as an incident signal) that enters the measurement target, and transmits an electromagnetic wave (also referred to as a reflected signal) reflected from the measurement target to the dielectric spectroscopy unit 12. The probes forming the ports other than the port 1 transmit, to the dielectric spectroscopy unit 12, an electromagnetic wave (also referred to as a transmitted signal) that has entered from the probe of the port 1 and then been transmitted (transferred) through the measurement target.

The dielectric spectroscopy unit 12 supplies the electromagnetic wave (an incident signal) that is to enter the measurement target to the probe 11, and receives the electromagnetic waves (reflected signals and transmitted signals) reflected from and transmitted through the measurement target via the probe 11. The dielectric spectroscopy unit 12 performs transform (Fourier transform) from a time-domain representation to a frequency-domain representation on each of the incident signal, the reflected signal, and the transmitted signal. As a result, frequency component signals representing the respective signals of the incident signal, the reflected signal, and the transmitted signal with frequency component values are calculated. The calculated frequency component signals of the incident signal, the reflected signal, and the transmitted signal are supplied to the signal processing unit 13.

The signal processing unit 13 calculates the S parameters of the measurement target, on the basis of the respective frequency component signals of the incident signal, the reflected signal, and the transmitted signal from the dielectric spectroscopy unit 12. For example, in a case where two ports of the vector network analyzer are used, S11 and S21 are calculated as the S parameters. S11 represents changes in magnitude and phase of the reflected signal with respect to the incident signal for the measurement target. S21 represents changes in magnitude and phase of the transmitted signal with respect to the incident signal for the measurement target. The calculated S parameters are supplied to the dielectric constant imaginary part calculation unit 14 and the moisture content calculation unit 15. Note that, in some cases, the S parameters may be calculated on the basis of the incident signal and the reflected signal among the incident signal, the reflected signal, and the transmitted signal. Further, in the present technology, the S parameters of the measurement target are calculated on the basis of the respective frequency component signals of the incident signal, the reflected signal, and the transmitted signal (alternatively, the incident signal and the reflected signal) from the dielectric spectroscopy unit 12, to calculate the value of the dielectric constant imaginary part and the moisture content of the measurement target. However, it is not always necessary to calculate the S parameters. For example, the value of the dielectric constant imaginary part of the measurement target and the moisture content may be calculated directly on the basis of the respective frequency component signals of the incident signal, the reflected signal, and the transmitted signal from the dielectric spectroscopy unit 12. Further, as for the method for calculating the complex dielectric constant of the measurement target, there are known method such as a probe method or an S-parameter method. By the probe method, the complex dielectric constant is calculated on the basis of S11, and, by the S-parameter method, the complex dielectric constant is calculated on the basis of S11 and S21. The present technology may be applied in cases where either one of the methods is used. For example, in a case where S11 and S21 are obtained as the S parameters, the complex dielectric constant may be calculated from S11 as in the probe method.

The dielectric constant imaginary part calculation unit 14 calculates the value of the imaginary part (the dielectric constant imaginary part) between the real part and the imaginary part in the complex dielectric constant of the measurement target, on the basis of the S parameters from the signal processing unit 13. The value of the dielectric constant imaginary part indicates the frequency characteristics depending on the concentration of glucose (glucose sugar) contained in the measurement target. The calculated value of the dielectric constant imaginary part is supplied to the glucose calculation unit 16.

Note that the complex dielectric constant ε* is expressed by Expression (1) shown below.

ε*=ε′−jε″ (1)

Here, ε′ represents the value of the real part of the complex dielectric constant, and ε″ represents the imaginary part value of the complex dielectric constant. The absolute value of the complex dielectric constant is the value of the square root of the sum of the squares of the value of the real part and the value of the imaginary part.

The moisture content calculation unit 15 calculates the absolute value (the dielectric constant absolute value) of the complex dielectric constant of the measurement target on the basis of the S parameters from the signal processing unit 13, and calculates the moisture content of the measurement target from a calibration curve between the dielectric constant absolute value and the volumetric moisture content, or from the ratio between the dielectric constant absolute value and the relative permittivity of moisture. The moisture content of the measurement target indicates the content of moisture in the substance contained in the measurement target. The calculated moisture content is supplied to the glucose calculation unit 16. Note that the moisture content calculation unit 15 may calculate the value of the dielectric constant real part or the delay time of the reflected signal with respect to the incident signal (the difference in reception time between the incident signal and the reflected signal), instead of the dielectric constant absolute value, and calculate the moisture content on the basis of a calibration curve between the value of the dielectric constant real part and the volumetric moisture content, or a calibration curve between the delay time of the reflected signal with respect to the incident signal and the volumetric moisture content.

The glucose calculation unit 16 calculates the glucose concentration (the blood glucose level, or the glucose concentration in interstitial fluid) in the measurement target, on the basis of the value of the dielectric constant imaginary part from the dielectric constant imaginary part calculation unit 14 and the moisture content from the moisture content calculation unit 15.

Note that the glucose concentration is also referred to as the glucose level. The unit of glucose concentration is the volume of glucose per unit volume. As for the frequency characteristics of the dielectric constant imaginary part, if the moisture content is different though the glucose concentration of the measurement target is the same, the frequency characteristics of the dielectric constant imaginary part are different. Therefore, in a case where a predetermined moisture content is assumed, for example, the glucose calculation unit 16 corrects the correction amount depending on the actual moisture content with respect to the glucose concentration estimated from the frequency characteristics of the dielectric constant imaginary part. The glucose calculation unit 16 determines the correction amount of the glucose concentration or the corrected glucose concentration, using a correction table prepared in advance. The correction table is data that specifies an appropriate correction amount for the glucose concentration or the corrected glucose concentration with respect to the glucose concentration estimated from the value (frequency characteristics) of the dielectric constant imaginary part and the actual moisture content from the moisture content calculation unit 15. The glucose concentration (glucose level) determined by the glucose calculation unit 16 is output to a processing unit in the subsequent stage. The processing unit in the subsequent stage may be a display control unit that controls display on a display unit, a management unit that manages (storing and the like of) data, or the like.

With the measurement device 1 to which the present technology is applied, the moisture content of the measurement target is calculated, so that the glucose concentration can be corrected depending on the moisture content, and the glucose concentration can be accurately calculated. Note that the glucose concentration may be measured by a method different from that used by the measurement device 1, with the use of infrared rays, visible light, or the like, for example, and the measurement result may be corrected on the basis of the moisture content calculated by the measurement device 1. Note that the present technology can also be applied in a case where the concentration of a substance (in vivo substance, for example) other than glucose in the measurement target is measured. In the measurement of the concentration of a substance other than glucose, an accurate concentration taking into account the moisture content can be measured in a manner similar to that used in a case where the concentration of glucose is measured.

### Example 1 of the Measurement Device 1

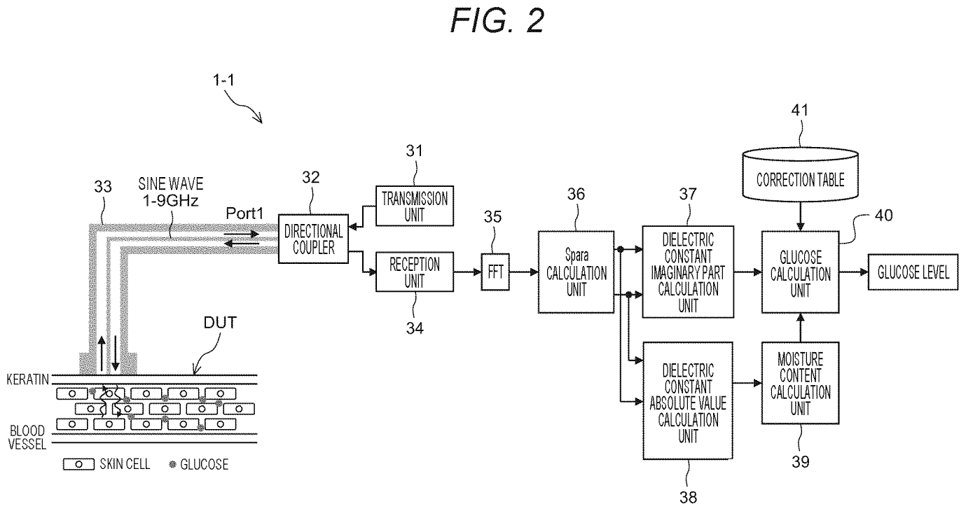

FIG. 2 is a configuration diagram illustrating an example configuration of a measurement device 1-1 that is Example 1 of the measurement device 1. The measurement device 1-1 in FIG. 2 is in a mode using only one port 1 of a vector network analyzer. The measurement device 1-1 in FIG. 2 includes a transmission unit 31, a directional coupling unit 32, a probe 33, a reception unit 34, an FFT unit 35, an S-parameter calculation unit 36, a dielectric constant imaginary part calculation unit 37, a dielectric constant absolute value calculation unit 38, a moisture content calculation unit 39, a glucose calculation unit 40, and a correction table 41. Note that, in FIGS. 2, 3, 4, and 12, “S-parameter calculation unit” is indicated as “Spara calculation unit”. The transmission unit 31, the directional coupling unit 32, the reception unit 34, and the FFT unit 35 in FIG. 2 are components of the dielectric spectroscopy unit 12 in FIG. 1. The probe 33 in FIG. 2 is a component of the probe 11 in FIG. 1. The S-parameter calculation unit 36 in FIG. 2 is a component of the signal processing unit 13 in FIG. 1. The dielectric constant imaginary part calculation unit 37 in FIG. 2 is a component of the dielectric constant imaginary part calculation unit 14 in FIG. 1. The dielectric constant absolute value calculation unit 38 and the moisture content calculation unit 39 in FIG. 2 are components of the moisture content calculation unit 15 in FIG. 1. The glucose calculation unit 40 and the correction table 41 in FIG. 2 are components of glucose calculation unit 16 in FIG. 1. A measurement target DUT is tissues around the portion with which an end portion (an electrode) of the probe 33 is brought into contact, such as an earlobe, an arm, a palm, a foot, or an abdomen of a person (or an animal). Note that, in FIG. 2, keratin, skin cells, and blood vessels (blood) are shown as body tissues included in the measurement target DUT, but the measurement target DUT is not limited to these tissues.

In FIG. 2, the transmission unit 31 supplies an electromagnetic wave (an incident signal) that is to enter the measurement target DUT, to the directional coupling unit 32. Here, the electromagnetic wave (the incident signal) is a signal of a fixed frequency or a signal obtained by sweeping a frequency. The directional coupling unit 32 divides and transfers the electromagnetic wave from the transmission unit 31 as an electromagnetic wave for the probe 33 and an electromagnetic wave for the reception unit 34. The directional coupling unit 32 also transfers the electromagnetic wave that is from the probe 33 and has been reflected from the measurement target DUT to the reception unit 34 (a reflected signal).

The probe 33 forms the port 1 of the vector network analyzer. The probe 33 irradiates (enters) the measurement target DUT with the electromagnetic wave (incident signal), which is from the transmission unit 31 and is supplied from the directional coupling unit 32, and transfers the electromagnetic wave (the reflected signal) reflected from the measurement target DUT to the directional coupling unit 32.

The reception unit 34 receives the input signal that is from the transmission unit 31 and is supplied from the directional coupling unit 32, and the reflected signal that is from the probe 33 and is supplied from the directional coupling unit 32, and supplies the signals to the FFT unit 35.

The FFT unit 35 performs transform (Fourier transform) from a time-domain representation to a frequency-domain representation on each of the input signal and the reflected signal from the reception unit 34, by performing a fast Fourier transform (FFT) process. By doing so, the FFT unit 35 calculates frequency component signals of the incident signal and the reflected signal, and supplies the frequency component signals to the S-parameter calculation unit 36.

The S-parameter calculation unit 36 calculates the S parameter of the measurement target DUT, on the basis of the incident signal and the reflected signal as the frequency component signals from the FFT unit 35. In the measurement device 1-1, only the incident signal and the reflected signal at the port 1 are supplied to the S-parameter calculation unit 36, and thus, S11 is calculated as the S parameter. S11 represents changes in magnitude and phase of the reflected signal with respect to the incident signal for the measurement target DUT. The S parameter calculated by the S-parameter calculation unit 36 is supplied to the dielectric constant imaginary part calculation unit 37 and the dielectric constant absolute value calculation unit 38.

The dielectric constant imaginary part calculation unit 37 calculates the value of the imaginary part (the dielectric constant imaginary part) between the real part and the imaginary part in the complex dielectric constant of the measurement target, on the basis of the S parameter from the S-parameter calculation unit 36. The value of the dielectric constant imaginary part indicates the frequency characteristics depending on the concentration of the glucose (glucose sugar) contained in the measurement target DUT. The calculated value of the dielectric constant imaginary part is supplied to the glucose calculation unit 40.

The dielectric constant absolute value calculation unit 38 calculates the absolute value (the dielectric constant absolute value) of the complex dielectric constant of the measurement target, on the basis of the S parameter from the S-parameter calculation unit 36. The dielectric constant absolute value varies depending on the content of moisture contained in the measurement target DUT. The calculated dielectric constant absolute value is supplied to the moisture content calculation unit 39.

The moisture content calculation unit 39 calculates the moisture content of the measurement target DUT, on the basis of the dielectric constant absolute value from the dielectric constant absolute value calculation unit 38. The calculated moisture content is supplied to the glucose calculation unit 40.

On the basis of the value of the dielectric constant imaginary part from the dielectric constant imaginary part calculation unit 37 and the moisture content from the moisture content calculation unit 39, the glucose calculation unit 40 calculates the glucose concentration (glucose level) in the measurement target DUT, using the correction table 41. That is, the glucose calculation unit 40 determines a correction amount corresponding to the glucose concentration estimated from the value (frequency characteristics) of the dielectric constant imaginary part and the moisture content from the moisture content calculation unit 39 in accordance with the correction table 41, and adds the determined correction amount to the glucose concentration estimated from the value of the dielectric constant imaginary part. The glucose level calculated by the glucose calculation unit 40 is output to a processing unit in the subsequent stage.

With the measurement device 1-1 according to Example 1, a glucose concentration can be measured by a so-called probe method, and the glucose concentration can be accurately calculated on the basis of the moisture content.

### Example 2 of the Measurement Device 1

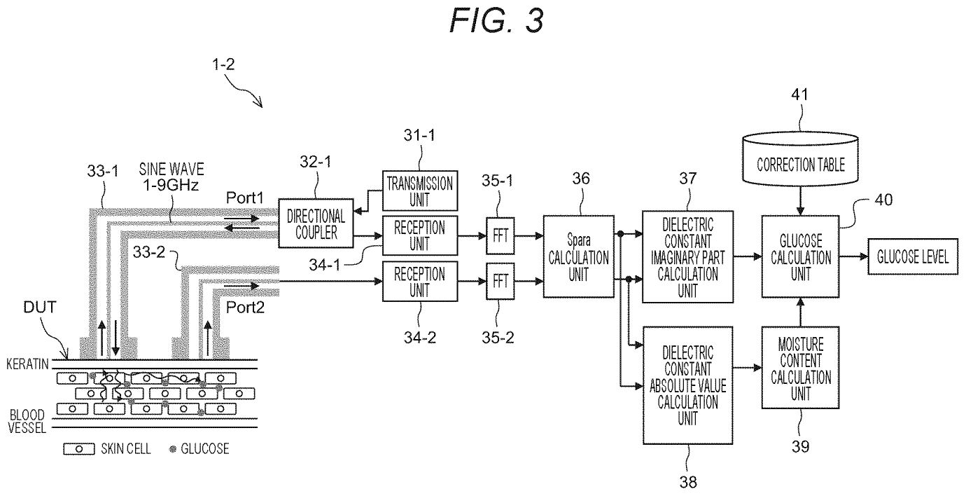

FIG. 3 is a configuration diagram illustrating an example configuration of a measurement device 1-2 that is Example 2 of the measurement device 1. The measurement device 1-2 in FIG. 3 is in a mode using the two of a port 1 and a port 2 of a vector network analyzer. Note that, in the drawing, the portions corresponding to those of the measurement device 1-1 in FIG. 2 are denoted by the same reference numerals, and explanation of them will be skipped as appropriate.

The measurement device 1-2 in FIG. 3 includes a transmission unit 31-1, a directional coupling unit 32-1, probes 33-1 and 33-2, reception units 34-1 and 34-2, FFT units 35-1 and 35-2, an S-parameter calculation unit 36, a dielectric constant imaginary part calculation unit 37, a dielectric constant absolute value calculation unit 38, a moisture content calculation unit 39, a glucose calculation unit 40, and a correction table 41. The transmission unit 31-1, the directional coupling unit 32-1, the reception units 34-1 and 34-2, and the FFT units 35-1 and 35-2 in FIG. 3 are components of the dielectric spectroscopy unit 12 in FIG. 1. The probes 33-1 and 33-2 in FIG. 3 are components of the probe 11 in FIG. 1. The S-parameter calculation unit 36 in FIG. 3 is a component of the signal processing unit 13 in FIG. 1. The dielectric constant imaginary part calculation unit 37 in FIG. 3 is a component of the dielectric constant imaginary part calculation unit 14 in FIG. 1. The dielectric constant absolute value calculation unit 38 and the moisture content calculation unit 39 in FIG. 3 are components of the moisture content calculation unit 15 in FIG. 1. The glucose calculation unit 40 and the correction table 41 in FIG. 3 are components of glucose calculation unit 16 in FIG. 1. A measurement target DUT is similar to the measurement target DUT in FIG. 2.

In FIG. 3, the transmission unit 31-1, the directional coupling unit 32-1, the probe 33-1, the reception unit 34-1, and the FFT unit 35-1 are similar to the transmission unit 31, the directional coupling unit 32, the probe 33-1, the reception unit 34, and the FFT unit 35 in FIG. 2, and therefore, explanation of them is not made herein.

The probe 33-2 forms the port 2 of the vector network analyzer. The probe 33-2 transfers an electromagnetic wave (a transmitted signal) transmitted (transferred) through the measurement target DUT from the transmission unit 31-1 to the reception unit 34-2.

The reception unit 34-2 receives the transmitted signal from the probe 33-2, and supplies the transmitted signal to the FFT unit 35-2.

The FFT unit 35-2 performs transform (Fourier transform) from a time-domain representation to a frequency-domain representation on the transmitted signal from the reception unit 34-2, by performing a fast Fourier transform (FFT) process. By doing so, the FFT unit 35-2 calculates a frequency component signal of the transmitted signal, and supplies the frequency component signal to the S-parameter calculation unit 36.

The S-parameter calculation unit 36 calculates the S parameters of the measurement target DUT, on the basis of an incident signal and a reflected signal as frequency component signals from the FFT unit 35-1, and the transmitted signal as the frequency component signal from the FFT unit 35-1. In the measurement device 1-2, the incident signal and the reflected signal at the port 1, and the transmitted signal at the port 2 are supplied to the S-parameter calculation unit 36, and thus, S11 and S21 are calculated as the S parameters. S11 represents changes in magnitude and phase of the reflected signal with respect to the incident signal for the measurement target DUT. S21 represents changes in magnitude and phase of the transmitted signal with respect to the incident signal for the measurement target DUT. The S parameter calculated by the S-parameter calculation unit 36 is supplied to the dielectric constant imaginary part calculation unit 37 and the dielectric constant absolute value calculation unit 38.

The dielectric constant imaginary part calculation unit 37 calculates the value of the imaginary part (the dielectric constant imaginary part) between the real part and the imaginary part in the complex dielectric constant of the measurement target, on the basis of the S parameters from the S-parameter calculation unit 36. Note that, in the measurement device 1-2, the complex dielectric constant can be calculated on the basis of S11 and S21, which are S parameters. Accordingly, the value of the dielectric constant imaginary part is also calculated on the basis of S11 and S21. The calculated value of the dielectric constant imaginary part is supplied to the glucose calculation unit 40.

The dielectric constant absolute value calculation unit 38 calculates the absolute value (the dielectric constant absolute value) of the complex dielectric constant of the measurement target, on the basis of the S parameter from the S-parameter calculation unit 36. Note that the dielectric constant absolute value is also calculated on the basis of S11 and S21. The calculated dielectric constant absolute value is supplied to the moisture content calculation unit 39.

The moisture content calculation unit 39 calculates the moisture content of the measurement target DUT, on the basis of the dielectric constant absolute value from the dielectric constant absolute value calculation unit 38. The calculated moisture content is supplied to the glucose calculation unit 40.

On the basis of the value of the dielectric constant imaginary part from the dielectric constant imaginary part calculation unit 37 and the moisture content from the moisture content calculation unit 39, the glucose calculation unit 40 calculates the glucose concentration (glucose level) in the measurement target DUT, using the correction table 41. The calculated glucose level is output to a processing unit in the subsequent stage.

With the measurement device 1-2 according to Example 2, a glucose concentration can be measured by a so-called S-parameter method, and the glucose concentration can be accurately calculated on the basis of the moisture content. In some cases, the glucose concentration may be calculated by the measurement device 1-2 using a probe method.

### Example 3 of the Measurement Device 1

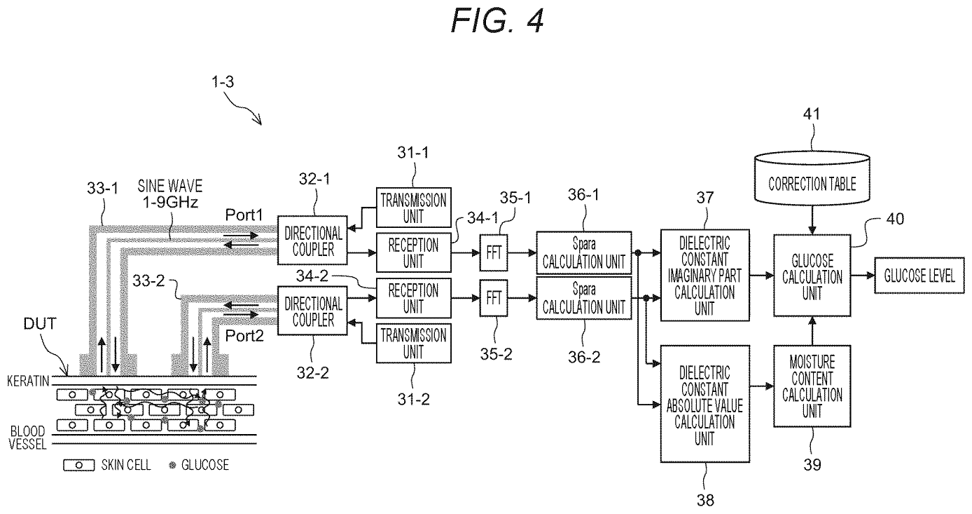

FIG. 4 is a configuration diagram illustrating an example configuration of a measurement device 1-3 that is Example 3 of the measurement device 1. The measurement device 1-3 in FIG. 4 is in a mode using the two of a port 1 and a port 2 of a vector network analyzer, and is a mode in which electromagnetic waves can enter in a time-division manner from both the ports 1 and 2. Note that, in the drawing, the portions corresponding to those of the measurement device 1-2 in FIG. 3 are denoted by the same reference numerals, and explanation of them will be skipped as appropriate.

The measurement device 1-3 in FIG. 4 includes transmission units 31-1 and 31-2, a directional coupling unit 32-1, probes 33-1 and 33-2, reception units 34-1 and 34-2, FFT units 35-1 and 35-2, S-parameter calculation units 36-1 and 36-2, a dielectric constant imaginary part calculation unit 37, a dielectric constant absolute value calculation unit 38, a moisture content calculation unit 39, a glucose calculation unit 40, and a correction table 41. The transmission units 31-1 and 31-2, the directional coupling units 32-1 and 32-2, the reception units 34-1 and 34-2, and the FFT units 35-1 and 35-2 in FIG. 4 are components of the dielectric spectroscopy unit 12 in FIG. 1. The probes 33-1 and 33-2 in FIG. 4 are components of the probe 11 in FIG. 1. The S-parameter calculation units 36-1 and 36-2 are components of the signal processing unit 13 in FIG. 1. The dielectric constant imaginary part calculation unit 37 in FIG. 4 is a component of the dielectric constant imaginary part calculation unit 14 in FIG. 1. The dielectric constant absolute value calculation unit 38 and the moisture content calculation unit 39 in FIG. 4 are components of the moisture content calculation unit 15 in FIG. 1. The glucose calculation unit 40 and the correction table 41 in FIG. 4 are components of glucose calculation unit 16 in FIG. 1. A measurement target DUT is similar to the measurement target DUT in FIG. 3. The transmission unit 31-1, the directional coupling unit 32-1, the probe 33-1, the reception unit 34-1, and the FFT unit 35-1 are similar to the transmission unit 31-1, the directional coupling unit 32-1, the probe 33-1, the reception unit 34-1, and the FFT unit 35-1 in FIG. 3, and therefore, explanation of them is not made herein.

Further, the transmission unit 31-2, the directional coupling unit 32-2, the probe 33-2, the reception unit 34-2, and the FFT unit 35-2 in FIG. 4 are similar to the transmission unit 31-1, the directional coupling unit 32-1, the probe 33-1, the reception unit 34-1, and the FFT unit 35-1 in FIG. 3, and therefore, explanation of them is not made herein.

Note that, in FIG. 4, for an incident signal supplied from the transmission unit 31-1 to the measurement target DUT, the probe 33-2, the reception unit 34-2, and the FFT unit 35-2 function as constituent units for a transmitted signal, like the probe 33-2, the reception unit 34-2, and the FFT unit 35-2 in FIG. 3. For an incident signal supplied from the transmission unit 31-2 to the measurement target DUT, the probe 33-1, the reception unit 34-1, and the FFT unit 35-1 function as constituent units for a transmitted signal, like the probe 33-2, the reception unit 34-2, and the FFT unit 35-2 in FIG. 3.

The directional coupling units 32-1 and 32-2 function as directional coupling units that divide incident signals from the respective transmission units 31-1 and 31-2, and reflected signals or transmitted signals from the measurement target DUT, and supply the signals to the reception units 34-1 and 34-2. As a result, the FFT unit 35-1 calculates the respective frequency component signals of the incident signal supplied from the transmission unit 31-1 to the measurement target DUT via the probe 33-1 of the port 1, the reflected signal supplied with respect to the incident signal via the probe 33-1, and the transmitted signal supplied via the probe 33-1 of the port 1 with respect to an input signal supplied from the transmission unit 31-2 to the measurement target DUT via the probe 33-2 of the port 2, and supplies the frequency component signals to the S-parameter calculation unit 36-1. The FFT unit 35-2 calculates the respective frequency component signals of the incident signal supplied from the transmission unit 31-2 to the measurement target DUT via the probe 33-2 of the port 2, the reflected signal supplied with respect to the incident signal via the probe 33-2, and the transmitted signal supplied via the probe 33-2 of the port 2 with respect to the input signal supplied from the transmission unit 31-1 to the measurement target DUT via the probe 33-1 of the port 1, and supplies the frequency component signals to the S-parameter calculation unit 36-2.

The S-parameter calculation unit 36-1 calculates S parameters of the measurement target DUT, on the basis of the incident signal, the reflected signal, and the transmitted signal as the frequency component signals from the FFT unit 35-1. In the measurement device 1-3, the reflected signal from the measurement target DUT at the port 1 and the transmitted signal from the port 2 to the port 1 are used, so that the S-parameter calculation unit 36-1 calculates S11 and S12 as the S parameters. The S parameters calculated by the S-parameter calculation unit 36-1 are supplied to the dielectric constant imaginary part calculation unit 37 and the dielectric constant absolute value calculation unit 38.

The S-parameter calculation unit 36-2 calculates S parameters of the measurement target DUT, on the basis of the incident signal, the reflected signal, and the transmitted signal as the frequency component signals from the FFT unit 35-2. In the measurement device 1-3, the reflected signal from the measurement target DUT at the port 2 and the transmitted signal from the port 1 to the port 2 are used, so that the S-parameter calculation unit 36-2 calculates S22 and S21 as the S parameters. Where S11 and S21 represent changes in magnitude and phase of the reflected signal and the transmitted signal with respect to the incident signal from the port 1 to the measurement target DUT, S22 and S12 represent changes in magnitude and phase of the reflected signal and the transmitted signal with respect to the incident signal from the port 2 to the measurement target DUT. S22 and S12 correspond to S11 and S21 in a case where the positions of contact of the probe 33-1 of the port 1 and the probe 33-2 of the port 2 with the measurement target DUT are interchanged.

The dielectric constant imaginary part calculation unit 37 calculates the value of the imaginary part (the dielectric constant imaginary part) between the real part and the imaginary part in the complex dielectric constant of the measurement target on the basis of the S parameters from the S-parameter calculation unit 36-1 and the S-parameter calculation unit 36-2, and supplies the calculated value to the glucose calculation unit 40. Note that, in the measurement device 1-3, the complex dielectric constant can be calculated on the basis of only S11, only S22, S11 and S21, or S22 and S12, which are S parameters from the S-parameter calculation unit 36-1 and the S-parameter calculation unit 36-2. The dielectric constant imaginary part calculation unit 37 may supply the value of the dielectric constant imaginary part calculated on the basis of any one or a plurality of S11 only, S22 only, S11 and S21, or S22 and S12 to the glucose calculation unit 40 in some cases, or may supply the value (an average value, for example) obtained by integrating the values of those dielectric constant imaginary parts, to the glucose calculation unit 40 in some cases. In the integration of the values of the dielectric constant imaginary parts, a value (an average, for example) obtained by integrating the respective values obtained from S11 and S22, or a value (an average value, for example) obtained by integrating the respective values obtained from S11 and S21, and S22 and S12 may be calculated as the value of the dielectric constant imaginary part.

The dielectric constant absolute value calculation unit 38 calculates the absolute value (the dielectric constant absolute value) of the complex dielectric constant of the measurement target on the basis of the S-parameters from the S-parameter calculation unit 36-1 and the S-parameter calculation unit 36-2, and supplies the calculated value to the moisture content calculation unit 39. Note that, like the dielectric constant imaginary part calculation unit 37, the dielectric constant absolute value calculation unit 38 can calculate the dielectric constant absolute value with a plurality of combinations of S parameters. For example, the dielectric constant absolute value calculation unit 38 may supply the dielectric constant absolute value calculated on the basis of S11 and S21 and the dielectric constant absolute value calculated on the basis of S21 and S12 to the moisture content calculation unit 39 in some cases, or may supply the value (the average value, for example) obtained by integrating those dielectric constant absolute values to the moisture content calculation unit 39 in some cases. In the integration of the dielectric constant absolute values, there may be cases where the dielectric constant absolute value is calculated, S11 being the value (the average, for example) obtained by integrating S11 and S22, S21 being the value (the average value, for example) obtained by integrating S21 and S12.

The moisture content calculation unit 39 calculates the moisture content of the measurement target DUT on the basis of the dielectric constant absolute value from the dielectric constant absolute value calculation unit 38, and supplies the moisture content to the glucose calculation unit 40. In a case where the dielectric constant absolute value calculated on the basis of S11 and S21, and the dielectric constant absolute value calculated on the basis of S22 and S12 are supplied, the moisture content calculation unit 39 may supply the respective moisture contents calculated on the basis of those dielectric constant absolute values to the glucose calculation unit 40, or may supply the value (the average value, for example) obtained by integrating those dielectric constant absolute values to the glucose calculation unit 40 in some cases.

On the basis of the value of the dielectric constant imaginary part from the dielectric constant imaginary part calculation unit 37 and the moisture content from the moisture content calculation unit 39, the glucose calculation unit 40 calculates the glucose concentration (glucose level) in the measurement target DUT, using the correction table 41, and outputs the glucose concentration to a processing unit in the subsequent stage. In a case where the value of the dielectric constant imaginary part calculated on the basis of S11 and S21, and the value of the dielectric constant imaginary part calculated on the basis of S22 and S12 are supplied, the glucose calculation unit 40 may integrate (average) the glucose levels calculated on the basis of the values of the respective dielectric constant imaginary parts. In a case where the moisture content calculated from the dielectric constant absolute value based on S11 and S21, and the moisture content calculated from the dielectric constant absolute value based on S22 and S12 are supplied, the glucose calculation unit 40 may integrate (average) the glucose levels calculated on the basis of the respective moisture contents.

With the measurement device 1-3 according to Example 3, a glucose concentration can be measured by a so-called S-parameter method, and the glucose concentration can be accurately calculated on the basis of the moisture contents. In some cases, the glucose concentration may be calculated by the measurement device 1-2 using a probe method.

### Second Embodiment of a Measurement Device to Which the Present Technology is Applied

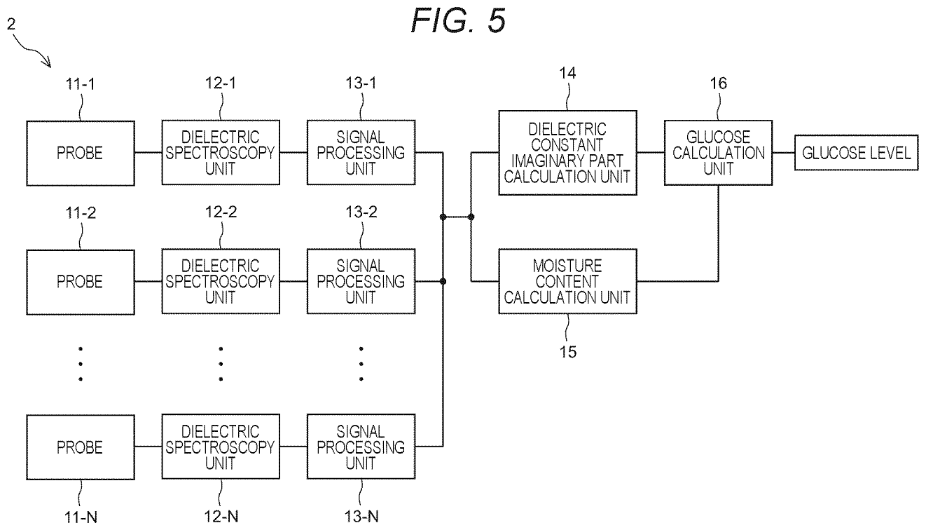

FIG. 5 is a block diagram illustrating an example schematic configuration of a second embodiment of a measurement device to which the present technology is applied. Note that, in the drawing, portions similar to those of the measurement device 1 in FIG. 1 are denoted by the same reference numerals, and explanation of them is not made herein.

In a measurement device 2 according to the second embodiment in FIG. 5 in comparison with the measurement device 1 according to the first embodiment in FIG. 1, the dielectric spectroscopy unit 12 and the signal processing unit 13 shown in FIG. 1 are provided for each N (N being a natural number of 2 or greater) probes 11-1 to 11-N (N being a natural number of 2 or greater) (for each N ports) included in the probe 11.

In FIG. 5, dielectric spectroscopy units 12-1 to 12-N and signal processing units 13-1 to 13-N perform processes equivalent to those to be performed by the dielectric spectroscopy unit 12 and the signal processing unit 13 in a case where one probe is used as the probe 11 in the measurement device 1 in FIG. 1. For example, Example 3 of the measurement device 1 in FIG. 4 corresponds to the mode of the measurement device 2 in FIG. 5 in a case where N is 2.

### Example of Processing Procedures to Be Carried Out by a Measurement Device to Which the Present Technology is Applied

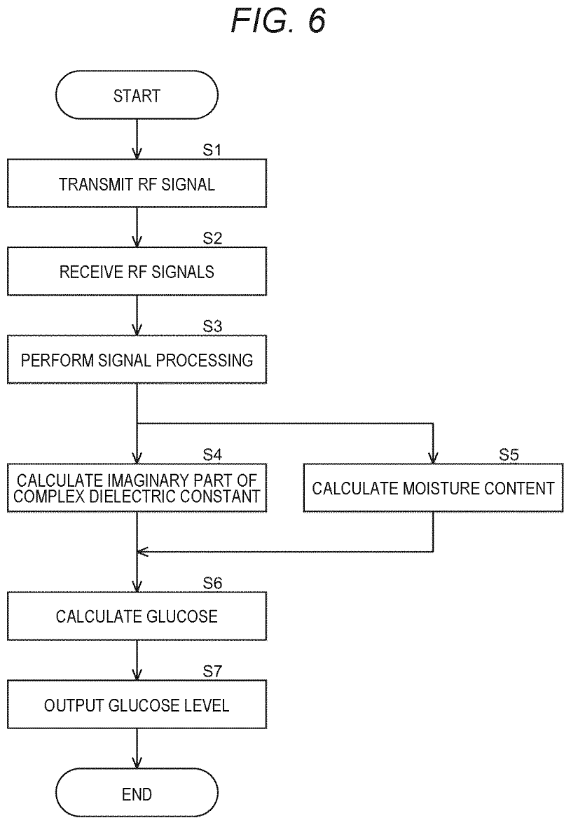

FIG. 6 is a flowchart illustrating an example of processing procedures to be carried out by a measurement device (the measurement device 1 in FIG. 1) to which the present technology is applied. In step S1, the dielectric spectroscopy unit 12 transmits an RF signal (an incident signal) to the measurement target via the probe 11. In step S2, the dielectric spectroscopy unit 12 receives RF signals (the incident signal and a reflected signal). Note that, in a case where two probes (ports) are used, a transmitted signal may also be received. In step S3, the dielectric spectroscopy unit 12 and the signal processing unit 13 perform signal processing on the received RF signals. As a result, an S parameter of the measurement target is calculated, and is supplied to the dielectric constant imaginary part calculation unit 14 and the moisture content calculation unit 15.

The process in step S4 and the process in step S5 are performed in parallel. In step S4, the dielectric constant imaginary part calculation unit 14 calculates the value (the frequency characteristics) of the imaginary part (the dielectric constant imaginary part) of the complex dielectric constant, on the basis of the S parameter calculated in step S3. In step S5, the moisture content calculation unit 15 calculates the absolute value (the dielectric constant absolute value) of the complex dielectric constant, on the basis of the S parameter calculated in step S3. In step S6, the glucose calculation unit 16 calculates the glucose level, on the basis of the value of the dielectric constant imaginary part calculated in step S4 and the moisture content calculated in step S5. That is, the glucose calculation unit 16 corrects the glucose level calculated from the value of the dielectric constant imaginary part, with a correction amount depending on the moisture content. In step S7, the glucose calculation unit 16 outputs the calculated glucose level to a processing unit in the subsequent stage. These processes in steps S1 to S7 are repeatedly performed.

### Example Configuration of the Dielectric Spectroscopy Unit in Example 2 of the Measurement Device 1

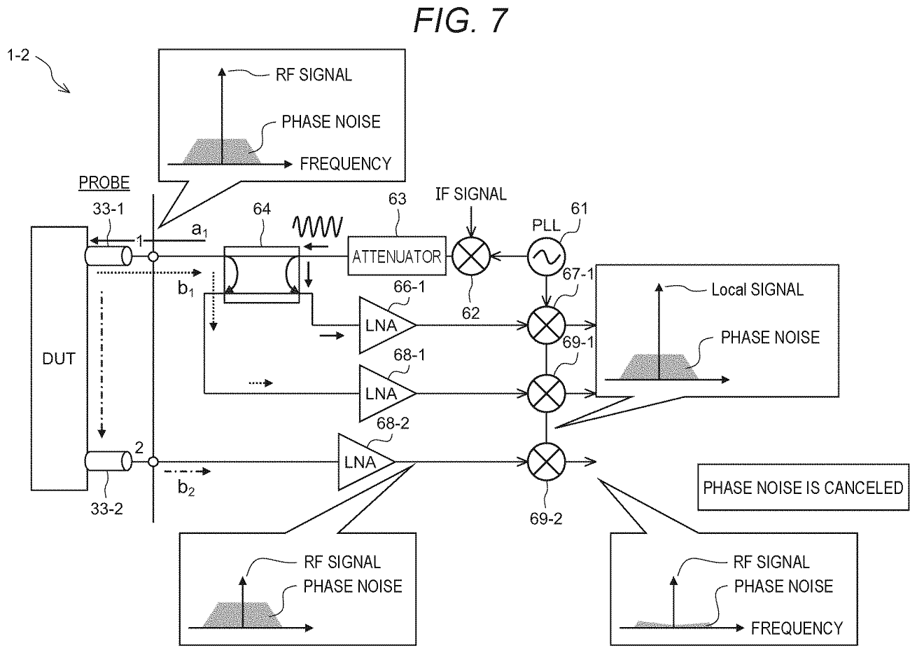

FIG. 7 is a configuration diagram illustrating an example of details of the dielectric spectroscopy unit 12 in the measurement device 1-2 according to Example 2 in FIG. 3. Note that FIG. 7 illustrates the configuration of the processing units in a stage before the FFT units 35-1 and 35-2 in FIG. 3.

In FIG. 7, the measurement device 1-2 includes the two probes 33-1 and 33-2 forming the ports 1 and 2, as in FIG. 3. In FIG. 7, the measurement device 1-2 includes a PLL circuit 61, mixers 62, 67-1, 69-1, and 69-2, an attenuator 63, a directional coupling unit 64, and low noise amplifiers (LNAs) 66-1, 68-1, and 68-2 as components of the dielectric spectroscopy unit 12. The PLL circuit 61 generates a radio-frequency reference clock, generates a local oscillation frequency signal (LO signal), and supplies the signals to the mixers 62, 67-1, 69-1, and 69-2. The mixer 62 performs frequency conversion by mixing an intermediate frequency (IF) signal with the LO signal, and generates an RF signal to be an input signal for the measurement target DUT. The RF signal has a higher frequency than the IF signal and the LO signal. The RF signal generated by the mixer 62 is supplied to the attenuator 63.

The attenuator 63 attenuates the intensity (the amplitude) of the RF signal from the mixer 62, and supplies the RF signal to the directional coupling unit 64. The directional coupling unit 64 divides the RF signal from the attenuator 63, supplies one RF signal to the probe 33-1 of the port 1, and supplies the other RF signal to the LNA 66-1. Also, the probe 33-1 supplies, as a reflected signal, the RF signal reflected by the measurement target DUT with respect to the incident signal, to the LNA 68-1. The RF signal that has entered the measurement target DUT as the incident signal from the probe 33-1 and been transmitted to the probe 33-2 of the port 2 is supplied as a transmitted signal to the LNA 68-2.

The LNA 66-1 supplies the RF signal received as the incident signal to the mixer 67-1. The LNA 68-1 supplies the RF signal received as the reflected signal to the mixer 69-1. The LNA 68-2 supplies the RF signal received as the transmitted signal to the mixer 69-2. The mixer 67-1 mixes the RF signal, which is the incident signal from the LNA 66-1, with the LO signal from the PLL circuit 61, to perform conversion into a frequency of an IF signal. The mixer 69-1 mixes the RF signal, which is the reflected signal from the LNA 68-1, with the LO signal from the PLL circuit 61, to perform conversion into a frequency of an IF signal. The mixer 69-2 mixes the RF signal, which is the transmitted signal from the LNA 68-2, with the LO signal from the PLL circuit 61, to perform conversion into a frequency of an IF signal. The incident signal, the reflected signal, and the transmitted signal converted into the IF signals by the mixers 67-1, 69-1, and 69-2 are supplied to the FFT units 35-1 and 35-2 in FIG. 3.

With the above configuration of the dielectric spectroscopy unit, in a case where noise (phase noise or the like) is present in the frequency component of the LO signal to be mixed with the IF signal in the mixer 62, the noise component is included in the RF signals, which are the incident signal, the reflected signal, and the transmitted signal. On the other hand, noise components such as phase noise in the IF signals generated by the mixers 67-1, 69-1, and 69-2 are removed by using a signal similar to the LO signal mixed in the mixer 62 as the LO signal to be mixed with an RF signal in the mixers 67-1, 69-1, and 69-2. Accordingly, highly accurate measurement can be performed with the low-power PLL circuit 61. Note that the example configuration illustrated in FIG. 7 can also be adopted in a measurement device having in a mode different from that of the measurement device 1-1 according to Example 2 in FIG. 3, such as the measurement device 1-2 according to Example 1 in FIG. 2, the measurement device 1-3 according to Example 3 in FIG. 4, and the measurement device 2 according to the second embodiment in FIG. 5.

### Arrangement of Probes with Respect to a Measurement Target

### <Example Arrangement 1>

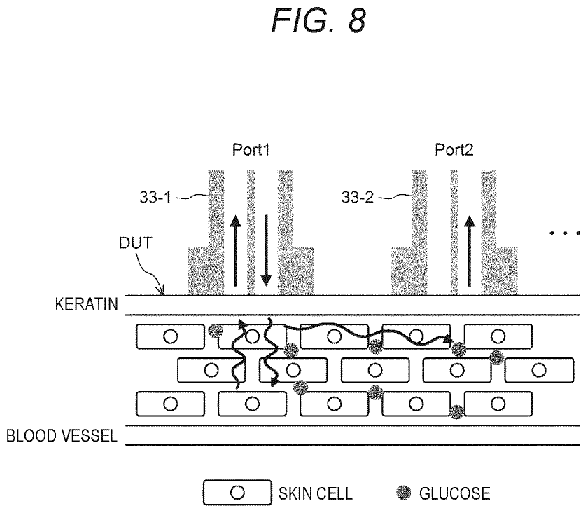

FIG. 8 is a diagram illustrating an example arrangement 1 of probes. In FIG. 8, probes 33-1 and 33-2 represent the probes 33-1 and 33-2 of the ports 1 and 2 shown in FIG. 3, for example. In FIG. 8, the probes 33-1 and 33-2 are arranged so that the surfaces of contact of the end portions (electrodes) thereof with the measurement target DUT are positioned along a plane. In a case where the number (plurality) of probes is a desired number other than two, the end portions (electrodes) of the probes can also be arranged at positions along a plane. Specifically, a plurality of electrodes including at least one of an electrode of a probe that causes an incident signal to enter the measurement target DUT and acquires a reflected signal, and an electrode of a probe that acquires a transmitted signal is arranged at positions along a plane. In this case, the lengths of the transmission lines for RF signals to be transmitted through the respective probes (ports) can be made uniform, and thus, correction of a delay caused by a difference in length between the transmission lines is unnecessary.

### <Example Arrangement 2>

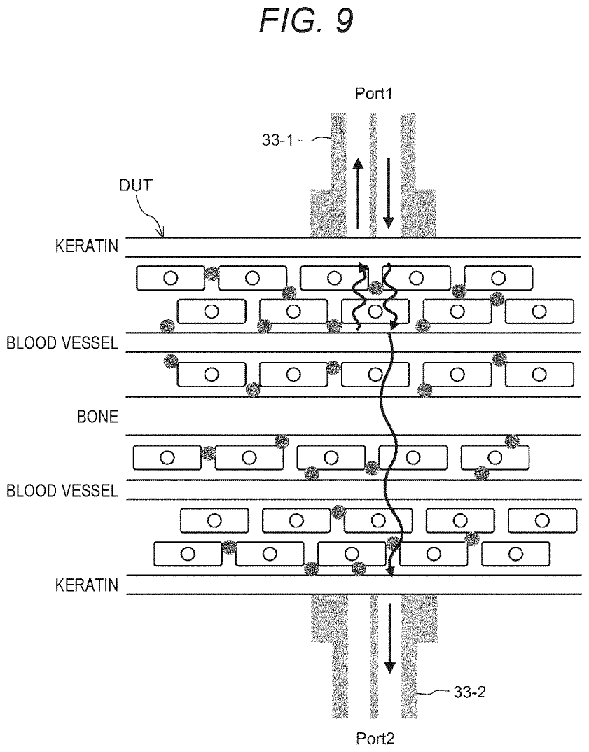

FIG. 9 is a diagram illustrating an example arrangement 2 of probes. In FIG. 9, probes 33-1 and 33-2 represent the probes 33-1 and 33-2 of the ports 1 and 2 shown in FIG. 3, for example. In FIG. 9, the probes 33-1 and 33-2 are arranged so that the surfaces of contact of the end portions (electrodes) thereof with the measurement target DUT are positioned to face each other. In a case where the number (plurality) of probes is a desired number other than two, the end portions (electrodes) of the probes can also be arranged at positions facing each other. Specifically, a plurality of electrodes including at least one of an electrode of a probe that causes an incident signal to enter the measurement target DUT and acquires a reflected signal, and an electrode of a probe that acquires a transmitted signal is arranged at positions facing each other. In a case where the measurement target DUT is a finger, an arm, or the like, the glucose level in a region in which not only skin cells and blood vessels (blood) near the surface of the skin but also a bone and the like exist is measured. In a case where electrodes of probes are arranged to face each other as in this example arrangement, the lengths of the transmission lines for RF signals to be transmitted through the respective probes (ports) can be made uniform, and accordingly, correction of a delay caused by a difference in length between the transmission lines is unnecessary.

### <Example of the Example Arrangement 1>

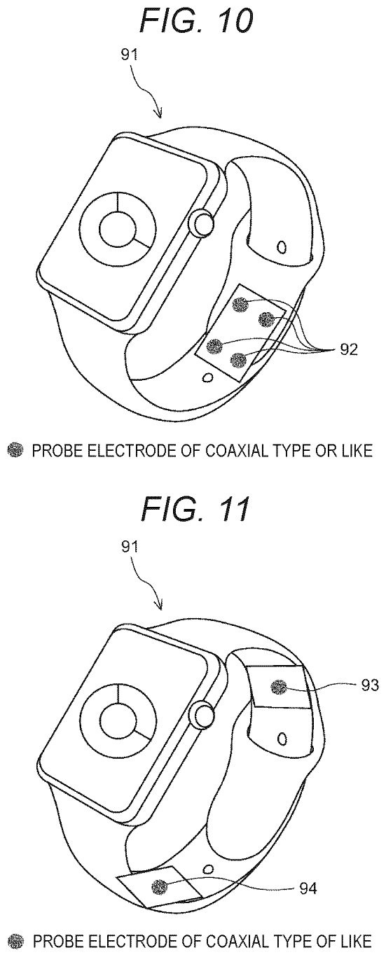

FIG. 10 is a diagram illustrating an example in which the example arrangement 1 of probes is adopted. In FIG. 10, a smartwatch 91 uses a band to attach its main body to the wrist. Note that the main body of the smartwatch 91 is mounted with the dielectric spectroscopy unit 12, the signal processing unit 13, and the like of the measurement device 1 illustrated in FIG. 1 and others. The smartwatch 91 is an example of a device in which a device main body on which processing units are mounted is attached to a measurement target with an attachment tool such as a band, and is not limited to any specific kind of device. In this case, a plurality of electrodes 92 of the plurality of probes of the measurement device 1 is arranged at positions along the plane of the inner surface of the band (the surface on the side in contact with the wrist). Also, the plurality of electrodes 92 is arranged at positions facing the main body on which the dielectric spectroscopy unit 12 and the signal processing unit 13 are mounted. As a result, the lengths of the transmission lines of the RF signals to be transmitted through the respective probes can be made uniform.

### <Example of the Example Arrangement 2>

FIG. 11 is a diagram illustrating an example in which the example arrangement 1 of probes is adopted. In FIG. 11, a smartwatch 91 is the same as that in FIG. 10. In FIG. 11, two electrodes 93 and 94 of the two probes of the measurement device 1 are arranged at opposite positions on the inner surface of the band (the surface on the side in contact with the wrist). Also, the electrodes 93 and 94 are arranged at positions at which the distance is transparent with respect to the main body of the smartwatch 91. As a result, the lengths of the transmission lines of the RF signals to be transmitted through the respective probes can be made uniform.

### Specific Example of Configurations of a Dielectric Spectroscopy Unit and a Signal Processing Unit

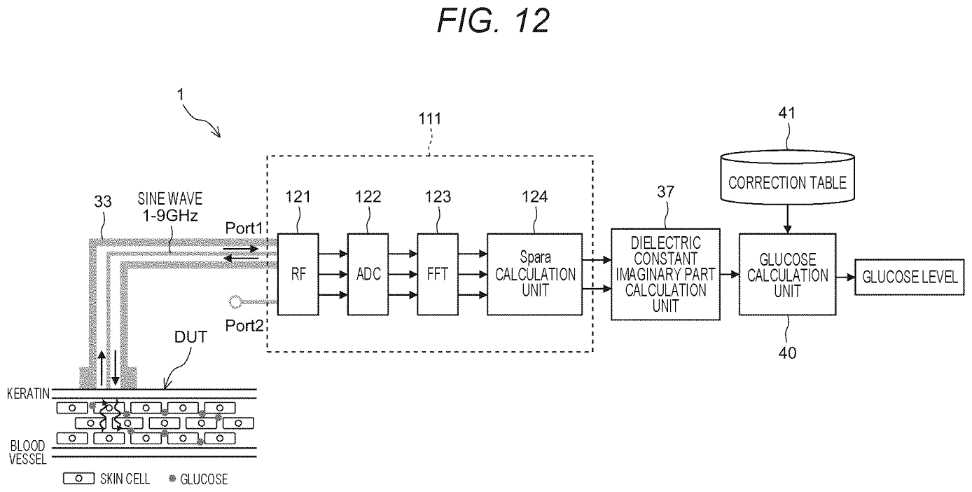

FIG. 12 is a diagram illustrating a specific example of configurations of the circuits (the dielectric spectroscopy unit 12 and the signal processing unit 13) before the S parameter is calculated in the measurement device 1 in FIG. 1. A measurement device 1 in FIG. 12 represents a mode in which a probe 33 of one port 1 is used as in the measurement device 1-1 according to Example 1 in FIG. 2. In the drawing, the same reference numerals are given to portions similar to those in FIG. 2, and explanation of them will not be made herein. Further, some blocks in FIG. 2 are omitted.

In FIG. 12, a circuit unit 111 represents some of the circuits disposed in the dielectric spectroscopy unit 12 and the signal processing unit 13, and includes an RF circuit 121, an analog to digital converter (ACD) 122, an FFT unit 123, and an S-parameter calculation unit 124.

The RF circuit 121 generates and outputs an RF signal having the frequency of an electromagnetic wave that is an incident signal to enter the measurement target DUT, receives RF signals that are the incident signal and a reflected signal, and performs frequency conversion into an IF signal or the like. The incident signal and the reflected signal received by the RF circuit 121 and subjected to the frequency conversion into an IF signal are supplied to the ADC 122.

The ADC 122 samples the incident signal and the reflected signal from the RF circuit 121 in a predetermined sampling cycle, converts the signals into digital data, and then supplies the digital data to the FFT unit 123. The FFT unit 123 performs FFT processing on the incident signal and the reflected signal converted into digital data by the ADC 122, and transforms the incident signal and the reflected signal from a time-domain representation into a frequency-domain representation. The FFT unit 123 calculates the S parameter on the basis of the incident signal and the reflected signal transformed into the frequency-domain representation, and supplies the S parameter to the dielectric constant imaginary part calculation unit 37 in the subsequent stage.

All or some of the RF circuit 121, the ACD 122, the FFT unit 123, and the S-parameter calculation unit 124 of the circuit unit 111 can be mounted on the same substrate or on one chip. Further, the configuration of the circuit unit 111 in FIG. 12 can also be applied to a measurement device for a purpose of use other than measurement of a glucose concentration.

### <Example Combinations of Configurations>

Note that the present technology may also provide the following configurations.
(1)

A measurement device including
a processing unit that calculates a glucose concentration in a measurement target, on the basis of a value of an imaginary part of a complex dielectric constant of the measurement target and a moisture content of the measurement target.
(2)

The measurement device according to claim 1, in which
the processing unit calculates the glucose concentration in the measurement target, on the basis of the value of the imaginary part of the complex dielectric constant and the moisture content of the measurement target.
(3)

The measurement device according to (1) or (2), in which
the processing unit calculates the moisture content, on the basis of at least an incident signal and a reflected signal among the incident signal as an electromagnetic wave that has entered the measurement target, the reflected signal reflected from the measurement target with respect to the incident signal, and a transmitted signal transmitted through the measurement target with respect to the incident signal.
(4)

The measurement device according to (3), in which
the processing unit calculates an absolute value or a value of a real part of the complex dielectric constant on the basis of at least the incident signal and the reflected signal among the incident signal, the reflected signal, and the transmitted signal, and calculates the moisture content on the basis of the absolute value or the value of the real part of the complex dielectric constant.
(5)

The measurement device according to (4), in which
the processing unit calculates the moisture content, using a calibration curve between the absolute value or the value of the real part of the complex dielectric constant, and the moisture content.
(6)

The measurement device according to (4) or (5), in which
the processing unit calculates the S parameter on the basis of at least the incident signal and the reflected signal among the incident signal, the reflected signal, and the transmitted signal, and calculates the absolute value or the value of the real part of the complex dielectric constant on the basis of the S parameter.
(7)

The measurement device according to (1) to (6), in which
the processing unit calculates the complex dielectric constant, on the basis of at least an incident signal and a reflected signal among the incident signal as an electromagnetic wave that has entered the measurement target, the reflected signal reflected from the measurement target with respect to the incident signal, and a transmitted signal transmitted through the measurement target with respect to the incident signal.
(8)

The measurement device according to (7), in which
the processing unit calculates the S parameter on the basis of at least the incident signal and the reflected signal among the incident signal, the reflected signal, and the transmitted signal, and calculates the imaginary part of the complex dielectric constant on the basis of the S parameter.
(9)

The measurement device according to any one of (3) to (8), further including
at least a first electrode between the first electrode that causes the incident signal to enter the measurement target and acquires the reflected signal, and a second electrode that acquires the transmitted signal, in which
   a plurality of electrodes including at least one of the first electrode or the second electrode is arranged at positions along a plane.
(10)

The measurement device according to any one of (3) to (8), further including
at least a first electrode between the first electrode that causes the incident signal to enter the measurement target and acquires the reflected signal, and a second electrode that acquires the transmitted signal, in which
a plurality of electrodes including at least one of the first electrode or the second electrode is arranged at positions facing each other, the measurement target being interposed between the positions.
(11)

The measurement device according to any one of (3) to (10), further including
a PLL circuit that generates an LO signal to be mixed with an IF signal to generate the incident signal, in which
the LO signal that is to be mixed with one of the reflected signal or the transmitted signal to generate the IF signal is generated by the PLL circuit.
(12)

A measurement method implemented by a processing unit of a measurement device including
the processing unit,
the measurement method including
   calculating a glucose concentration in a measurement target, on the basis of a complex dielectric constant of the measurement target and a moisture content of the measurement target.
(13)

A measurement device including:
an RF circuit that generates an incident signal as an electromagnetic wave to enter a measurement target, and receives the incident signal and a reflected signal reflected from the measurement target with respect to the incident signal;
an AD converter that converts the incident signal and the reflected signal received by the RF circuit into digital data;
a transform unit that performs Fourier transform on the incident signal and the reflected signal converted into the digital data by the AD converter; and
an S-parameter calculation unit that calculates an S parameter, on the basis of the incident signal and the reflected signal subjected to the Fourier transform by the transform unit.
(14)

The measurement device according to (13), in which
the RF circuit, the AD converter, the transform unit, and the S-parameter calculation unit are mounted on a chip.
(15) The measurement device according to (13) or (14), in which the S parameter is used for measuring a glucose concentration in the measurement target.
(16)

A measurement method implemented by a measurement device including:
an RF circuit;
an AD converter;
a transform unit; and
an S-parameter calculation unit,
the measurement method including:
   the RF circuit generating an incident signal as an electromagnetic wave to enter a measurement target, and receiving the incident signal and a reflected signal reflected from the measurement target with respect to the incident signal;
   the AD converter converting the incident signal and the reflected signal received by the RF circuit into digital data;
   the transform unit performing Fourier transform on the incident signal and the reflected signal converted into the digital data by the AD converter; and
   the S-parameter calculation unit calculating an S parameter on the basis of the incident signal and the reflected signal subjected to the Fourier transform by the transform unit.

Note that the present embodiment is not limited to the embodiments described above, and various modifications can be made without departing from the gist of the present disclosure. Furthermore, the effects described in the present specification are merely examples and are not restrictive, and some other effects may be achieved.

### REFERENCE SIGNS LIST

1, 2 Measurement device
11 Probe
12 Dielectric spectroscopy unit
13 Signal processing unit
14 Dielectric constant imaginary part calculation unit
15 Moisture content calculation unit
16 Glucose calculation unit
31 Transmission unit
32 Directional coupling unit
33 Probe
34 Reception unit
35 FFT unit
36 S-parameter calculation unit
37 Dielectric constant imaginary part calculation unit
38 Dielectric constant absolute value calculation unit
39 Moisture content calculation unit
40 Glucose calculation unit
41 Correction table
61 PLL circuit
62 Mixer
63 Attenuator
64 Directional coupling unit

What is claimed is:

1. A measurement device, comprising
a processing unit that calculates a glucose concentration in a measurement target, on a basis of a complex dielectric constant of the measurement target and a moisture content of the measurement target.

2. The measurement device according to claim 1, wherein
the processing unit calculates the glucose concentration in the measurement target, on a basis of a value of an imaginary part of the complex dielectric constant and the moisture content of the measurement target.

3. The measurement device according to claim 1, wherein
the processing unit calculates the moisture content, on a basis of at least an incident signal and a reflected signal among the incident signal as an electromagnetic wave that has entered the measurement target, the reflected signal reflected from the measurement target with respect to the incident signal, and a transmitted signal transmitted through the measurement target with respect to the incident signal.

4. The measurement device according to claim 3, wherein
the processing unit calculates an absolute value or a value of a real part of the complex dielectric constant on a basis of at least the incident signal and the reflected signal among the incident signal, the reflected signal, and the transmitted signal, and calculates the moisture content on a basis of the absolute value or the value of the real part of the complex dielectric constant.

5. The measurement device according to claim 4, wherein
the processing unit calculates the moisture content, using a calibration curve between the absolute value or the value of the real part of the complex dielectric constant, and the moisture content.

6. The measurement device according to claim 4, wherein
the processing unit calculates the S parameter on a basis of at least the incident signal and the reflected signal among the incident signal, the reflected signal, and the transmitted signal, and calculates the absolute value or the value of the real part of the complex dielectric constant on a basis of the S parameter.

7. The measurement device according to claim 1, wherein
the processing unit calculates the complex dielectric constant, on a basis of at least an incident signal and a reflected signal among the incident signal as an electromagnetic wave that has entered the measurement target, the reflected signal reflected from the measurement target with respect to the incident signal, and a transmitted signal transmitted through the measurement target with respect to the incident signal.

8. The measurement device according to claim 7, wherein
the processing unit calculates the S parameter on a basis of at least the incident signal and the reflected signal among the incident signal, the reflected signal, and the transmitted signal, and calculates the imaginary part of the complex dielectric constant on a basis of the S parameter.

9. The measurement device according to claim 3, further comprising
at least a first electrode between the first electrode that causes the incident signal to enter the measurement target and acquires the reflected signal, and a second electrode that acquires the transmitted signal, wherein
a plurality of electrodes including at least one of the first electrode or the second electrode is arranged at positions along a plane.

10. The measurement device according to claim 3, further comprising
at least a first electrode between the first electrode that causes the incident signal to enter the measurement target and acquires the reflected signal, and a second electrode that acquires the transmitted signal, wherein
a plurality of electrodes including at least one of the first electrode or the second electrode is arranged at positions facing each other, the measurement target being interposed between the positions.

11. The measurement device according to claim 3, further comprising
a PLL circuit that generates an LO signal to be mixed with an IF signal to generate the incident signal, wherein
the LO signal that is to be mixed with one of the reflected signal or the transmitted signal to generate the IF signal is generated by the PLL circuit.

12. A measurement method implemented by a processing unit of a measurement device, including
the processing unit,
the measurement method comprising
calculating a glucose concentration in a measurement target, on a basis of a complex dielectric constant of the measurement target and a moisture content of the measurement target.

13. A measurement device comprising:
an RF circuit that generates an incident signal as an electromagnetic wave to enter a measurement target, and receives the incident signal and a reflected signal reflected from the measurement target with respect to the incident signal;
an AD converter that converts the incident signal and the reflected signal received by the RF circuit into digital data;
a transform unit that performs Fourier transform on the incident signal and the reflected signal converted into the digital data by the AD converter; and
an S-parameter calculation unit that calculates an S parameter, on a basis of the incident signal and the reflected signal subjected to the Fourier transform by the transform unit.

14. The measurement device according to claim 13, wherein
the RF circuit, the AD converter, the transform unit, and the S-parameter calculation unit are mounted on a chip.

15. The measurement device according to claim 13, wherein
the S parameter is used for measuring a glucose concentration in the measurement target.

16. A measurement method implemented by a measurement device, including:
an RF circuit;
an AD converter;
a transform unit; and
an S-parameter calculation unit,
the measurement method comprising:
the RF circuit generating an incident signal as an electromagnetic wave to enter a measurement target, and receiving the incident signal and a reflected signal reflected from the measurement target with respect to the incident signal;
the AD converter converting the incident signal and the reflected signal received by the RF circuit into digital data;
the transform unit performing Fourier transform on the incident signal and the reflected signal converted into the digital data by the AD converter; and
the S-parameter calculation unit calculating an S parameter on a basis of the incident signal and the reflected signal subjected to the Fourier transform by the transform unit.
